(19) United States
(12) Patent Application Publication
(10) Pub. No.: US 2022/0023303 A1
(43) Pub. Date: Jan. 27, 2022

(54) USE OF RIBOFLAVIN TO BENEFIT BOWEL HEALTH

(71) Applicant: DSM IP Assets B.V., Heerlen (NL)

(72) Inventors: Igor BENDIK, Kaiseraugst (CH); Julia BIRD, Kaiseraugst (CH); Susanne DOLD-HÖRMANSPERGER, Kaiseraugst (CH)

(21) Appl. No.: 17/311,742

(22) PCT Filed: Dec. 9, 2019
(86) PCT No.: PCT/EP2019/084220

§ 371 Date: Jun. 8, 2021

(30) Foreign Application Priority Data
Dec. 11, 2018 (EP) 18211459.5

(51) Int. Cl. A61K 31/525; A23L 33/15; A23L 33/21; A23L 33/00; A61P 1/10; A61P 1/12

(52) U.S. Cl. CPC A61K 31/525; A23L 33/15; A23L 33/21; A23V 2002/00; A61P 1/10; A61P 1/12; A23L 33/40

(57) ABSTRACT

Riboflavin can have a beneficial effect in supporting bowel health in healthy people. Specifically it can relieve diarrhea or constipation, regulate bowel movement frequency, and regulating bowel movement urgency. The dosage brings the daily intake of riboflavin (consumed by diet and by supplement) above the RDA.

### BRIEF DESCRIPTION OF THE INVENTION

This invention relates to the use of riboflavin to regulate bowel movements, particularly in healthy people who are subject to constipation and/or diarrhea.

### BACKGROUND

Mild functional gastrointestinal disorders characterized by abdominal pain and discomfort, altered bowel habit to predominantly constipation- or diarrhea-type, and bowel movement urgency are widespread. While they are not life-threatening, these symptoms can nevertheless cause a significant impact on quality of life for millions of people globally. Chronic constipation affects around 12% of the global population and irritable bowel syndrome prevalence is approximately 11% worldwide. Public health costs of mild functional gastrointestinal disorders are considerable, resulting from visits to primary care providers, prescription and over-the-counter medication, and workplace absences.

The causes of functional gastrointestinal disorders have not yet been fully elucidated, however there are several promising leads. Chronic inflammation, particularly in the intestinal mucosa, is implicated. Certain genes appear to increase risk of developing gastrointestinal disorders. Gut-brain axis dysfunction may also be important. Furthermore, the gut microbiome may help in reducing inflammation and maintaining intestinal epithelium integrity, thus improving gut barrier function and potential playing a pivotal role in conditions such as irritable bowel syndrome.

It would be desirable to provide a nutraceutical or pharmaceutical which could help regulate bowel activity in healthy people who are not experiencing constipation and/or diarrhea and for those who do.

### DETAILED DESCRIPTION OF THE INVENTION

It has been found, in accordance with this invention that there is a correlation between the intake of riboflavin and bowel regulation, particularly in certain groups of people, such as those who are healthy, but are experiencing either diarrhea or constipation. As riboflavin intake increased, the most common stool type changed towards a more normal type and moved away from either diarrhea or constipation (FIG. 1), bowel motion frequency was more likely to be normal (FIG. 2), and bowel motion urgency normalized (FIG. 4) as riboflavin intake increased.

Thus, one embodiment of this invention is a method of promoting bowel health comprising administering an effective amount of riboflavin to a healthy person either experiencing diarrhea or constipation, or who is at risk of experiencing diarrhea or constipation. Another embodiment of this invention is the use of riboflavin in the manufacture of a medicament or nutraceutical or food to promote bowel health in a healthy person experiencing either diarrhea or constipation or who is at risk of experiencing diarrhea or constipation. Another embodiment is the non-therapeutic use of riboflavin to promote bowel health in a healthy person experiencing diarrhea or constipation.

Another embodiment of this invention is a method of relieving the symptoms of diarrhea or constipation experienced by a healthy person comprising administering an effective amount of riboflavin to the person in need thereof. Also another embodiment is the non-therapeutic use of riboflavin to relieve the symptoms of diarrhea or constipation experienced by a healthy person. Yet another embodiment is the use of riboflavin in the manufacture of a medicament or nutraceutical or food to relieve the symptoms of diarrhea or constipation in a healthy person.

Another embodiment of this invention is a method of regulating bowel movement frequency in a healthy person comprising administering riboflavin to a healthy person experiencing abnormal bowel movement frequency or is at risk of experiencing abnormal bowel movement frequency. Another embodiment is the non-therapeutic use of riboflavin to regulate bowel movement frequency in a healthy person. Another embodiment is the use of riboflavin in the manufacture of a medicament, nutraceutical or food to regulate bowel movement frequency in a healthy person.

Another embodiment of this invention is a method of normalizing bowel motion urgency comprising administering an effective amount of riboflavin to a healthy person in need of normalizing bowel motion urgency. Another embodiment is the non-therapeutic use of riboflavin to normalize bowel motion frequency in a healthy person. Another embodiment is the use of riboflavin to manufacture a medicament food or nutraceutical to normalize bowel motion frequency in a normal person.

Another embodiment of this invention is a method of normalizing stool type in a healthy person, comprising administering an effective amount of riboflavin to a healthy person in need thereof. Another embodiment is the non-therapeutic use of riboflavin to normalize stool type in a healthy person. Another embodiment is the use of riboflavin in the manufacture of a medicament, food, or nutraceutical to normalize stool type in a healthy person.

### Definitions

As used throughout the specification and claims, the following definitions apply: “Promoting bowel health” means to support the normal functioning of the bowel, including attaining normal bowel motion frequency and bowel motion consistency.

“Mild functional gastrointestinal disorder” means an abnormal functioning of the gastrointestinal tract that results in altered bowel habits producing mild discomfort not caused by structural or biochemical anomalies, and excluding irritable bowel syndrome. Some of the factors involved in the GI tract and its motility include: eating a low fibre diet, not enough exercise, traveling or other changes in routine, consuming a large amount of dairy products, stress, resisting the urge to have a bowel movement, resisting the urge to have a bowel movement due to hemorrhoid pain, overusing laxatives/stool softeners which can weaken the bowel muscles, consumption of antacids containing calcium and/or aluminum, taking medicines which are known to have such an effect (certain antidepressants, iron pills, and some narcotics), and pregnancy.

“Healthy person” means a person who has not been diagnosed with a disease nor condition which is characterized by constipation or diarrhea, including irritable bowel syndrome (IBS), Crohn's disease, sprue, or the like. A healthy person may experience constipation or diarrhea (mild functional gastrointestinal disorder) which is not IBS, Crohn's disease or sprue.

### DESCRIPTION OF THE FIGURES

FIG. 1 is a graph showing self-reported stool type and quartiles of riboflavin intake.

FIG. 2 shows self-reported proportion of subjects with normal bowel movement frequency in quartiles of riboflavin intake, in U.S. adults aged ≥19 years.

FIG. 3 shows self-reported bowel motion frequency and total riboflavin intake, stratified for fiber intake in U.S. adults aged ≥19 years. Data are means.

FIG. 4 shows self-reported urgency to empty the bowels according to total riboflavin intake: proportion of U.S. adults aged ≥19 years

### DOSAGES

The amount of riboflavin which should be consumed by an adult per day according to this invention is an amount greater than 3.1 mg. The current recommended daily amount (RDA) for healthy adults is in the range 1.1 to 1.6 mg per day depending on gender, and pregnancy and lactation status, which is usually obtained through a balanced diet. Thus, the total amount of riboflavin consumed per day (greater than 3.1 mg) includes both the amount consumed in the diet and a supplemented amount. Thus in one embodiment of this invention, the “effective amount” is a supplementation of at least 1-5 mg per day, which should be taken in addition to the 1.1 to 1.6 mg per day RDA which is derived from a normal diet. In preferred embodiments, the amount in the supplement is at least 1 to 3 mg above the RDA, and in more preferably at least 1.5 to 2 mg above the RDA. The upper limit of the amount of riboflavin which can be consumed has not been determined, as unused riboflavin is excreted by the body. Controlled clinical studies have not shown adverse effects with very high doses given.

Thus, the riboflavin may be taken in a single dose, or may be split into several doses to be taken throughout the day.

Riboflavin and Fibre Combination

Another embodiment of this invention is the combination of riboflavin and dietary fibre. Increases in bowel motion frequency were primarily found when fibre intakes were in the upper three quartiles (above 9 g/day). Most dieticians would consider a daily intake of 9 grams of fibre as low, and would point to at least 18 grams per day as the recommended amount. Thus, one embodiment of this invention is a daily dosage of supplements comprising a combination of an effective amount of riboflavin and, either together or separately, sufficient fibre to result in a person's daily consumption to be at least 18 grams per day.

The amount of additional fibre needed will, of course, depend on a person's individual intake of fibre from food. Our studies showed that the median intake was 13 grams per day. Thus one preferred embodiment is addition of at least 5 grams of fibre per day in addition to at least 13 grams of fibre per day from food, in other preferred embodiments the amount of supplemental fibre is at least 1, 2, 3, 4, 5, 6, 7, 8, 9, 10, 11, 12, 13, 14, 15, 16, 17, or 18 grams per day, such that the total amount of fibre is at least 18 grams per day. For people consuming a lower than median amount of fibre, one dosage may be 8 grams per day. Conversely, for people consuming a higher than median amount of fibre, a dosage may be 2 grams per day.

Thus, another embodiment of this invention is a composition comprising an effective amount of riboflavin in combination of dietary fibre. The amount of dietary fibre is at least 4 grams of fibre, preferably at least 5 grams per day more. There are numerous commercially available sources of fibre supplements.

Some examples of riboflavin and fibre dosages in a supplement are:
A. for people consuming a full RDA amount of riboflavin and a median amount of fibre per day: 1-2 mg riboflavin and 5 grams fibre.
B. for people consuming a full RDA amount of riboflavin and a lower than median amount of fibre per day: 1-2 mg riboflavin and 8 grams fibre.
C. for people consuming a full RDA amount of riboflavin and a higher than median amount of fibre per day: 1-2 mg riboflavin and 3 grams fibre.

Dosage Forms

The dosage form may be any convenient oral form. For the purposes of the invention, riboflavin and/or fibre is suitably provided in compositions for oral administration which may be solid or liquid galenical formulations, dietary compositions, pharmaceuticals, or food. Examples of solid galenical formulations are tablets, capsules (e.g. hard or soft shell gelatin capsules), pills, sachets, powders, granules and the like which contain the riboflavin and/or fibre together with conventional galenical carrier. Any conventional carrier material can be used. The carrier material can be organic or inorganic inert carrier material suitable for oral administration. Suitable carriers include water, gelatin, gum arabic, lactose, starch, magnesium stearate, talc, vegetable oils, and the like. Additionally additives such as flavouring agents, preservatives, stabilizers, emulsifying agents, buffers and the like may be added in accordance with accepted practices of pharmaceutical compounding. Additional active ingredients for coadministration with the riboflavin and/or fibre may administered, together in a single composition, or may be administered in individual dosage units. Dietary compositions comprising riboflavin and/or fibre can be beverages, instant beverages, or food supplements.

The following non-limiting Examples are presented to better illustrate the invention.

### Examples

Study Cohort

Data were obtained from NHANES, a representative survey that measures the health and nutritional status of the U.S. non-institutionalized population. Research protocols were reviewed and approved by the National Center for Health Statistics Research Ethics Review Board (protocol #2005-2006). Ethical review of the protocols used in NHANES protocols is conducted annually, with ongoing changes submitted through an amendment process. All participants gave written consent before participation. Information that could potentially identify participants is removed before data is made publicly available.

For this analysis, data from adults aged 20 years and over in the survey years 2005-2010 were selected. Participants were selected using a complex, multi-stage probability sampling design to produce a representative sample. Demographic information (gender, age, ethnicity, education, poverty-income ratio) was provided by self-report during an interview conducted by a trained interviewer in the participant's home. Questions relating to Bowel Health were administered to adults aged 20 years and older in the mobile examination center by means of a computer-assisted personal interviewing system. Dietary assessment was conducted by means of two 24-hour dietary recalls, the first conducted in-person at the mobile examination center, and the second conducted by telephone 3 to 10 days after the dietary interview but not on the same day of the week. Pregnancy status in women aged 20 to 44 years was determined by self-report; negative responses were confirmed with a urinary pregnancy test. Dietary supplement riboflavin intake in the 2005-2006 survey cycle was based on reported dietary supplement use over the previous 30 days, and calculated by modifying the program used to estimate calcium intake from supplements for these survey years. For the survey cycles 2007-2010, the total supplemental intake based on the 30 days questionnaire was also used, based on the totals provided in the dataset. Subjects who self-reported inflammatory bowel disease were excluded.

The Bowel Health questionnaire in survey cycles 2005-2006 and 2007-2008 consisted of six questions: four relate to the assessment of the Fecal Incontinence Severity Index (Rockwood, et al 1999 Dis Colon Rectum 42, 1525-1532) one question requests participants' self-reported weekly bowel motion frequency, and one question was about normal stool type on the Bristol Stool Scale (Lewis et al 1997 Scand J Gastroenterol 32: 920-924). A Fecal Incontinence Intensity Score (FISI) was calculated and binned into four categories using the RANK procedure to obtain approximately equal categories. In the 2009-2010 survey cycle, questions about bowel evacuation urgency, constipation and diarrhea incidence over the previous year, and laxative use over the past 30 days, were added.

Statistical Analyses

Statistical analyses were performed using SAS version 9.3 (SAS Institute Inc., Cary, N.C., USA). Statistical significance was set at 0.05. Means and percentages were adjusted to be representative of the U.S. population using the sample weights for the Day 2 Dietary dataset for analyses involving riboflavin intake, and Mobile Examination Center sample weights for other analyses (table 1), and accounting for the complex survey design using the SURVEYFREQ and SURVEYMEANS procedures.

Linear regression model parameters were divided into chunks representing socio-demographic, and lifestyle factors, similar to the approach used by Sternberg et al 2013 J. Nutr 143:948S-956S, and using bowel-health related factors identified by Mitsuhashi et al. 2017 Am J Gastroenterol 10.1038/ajg.2017.213 to investigate the relationship between riboflavin intake from foods and/or dietary supplements on bowel motion frequency. Gender, ethnicity, education, age (continuous), Poverty-Income Ratio (PIR, continuous) were included in the socio-demographic chunk, and vigorous physical activity over the past 30 days (Y/N), dietary supplement use (Y/N), fiber and moisture intake (continuous), self-report of thyroid problems (Y/N) and use of prescription medication (1 or less vs 2 or more) were included in the diet and health chunk.

Stepwise linear regression model selection was first performed with the REG procedure to identify consistently non-significant predictors in the model chunks and then combined into the full model: based on these results, vigorous physical activity and dietary supplement use were removed. The final linear regression using significant predictors was performed using SURVEYREG to generate the correct variance estimation that takes the complex survey design into account.

Results

In total, 17,110 adults aged 20 and over were selected from NHANES survey cycles 2005-2010 to make up the analysis dataset.

Table 1 presents demographics, socio-economic parameters, pregnancy status, dietary intakes and supplement usage, laxative and prescription medication use, and bowel health parameters. Laxative use was only recorded for one third of the dataset, survey years 2009-2010. No significant differences between survey years were found.

TABLE 1
Participant demographic, lifestyle, dietary and bowel health characteristics for adults ≥20 y, NHANES 2005-2010

| | 2005-2006 | | | 2007-2008 | | | 2009-2010 | | | Entire dataset | | |
|---|---|---|---|---|---|---|---|---|---|---|---|---|
| Variable | N | Value | | N | Value | | N | Value | | N | Value | |
| Age (mean [SE], y) | 5033 | 46.1 | [0.73] | 5858 | 46.3 | [0.48] | 6219 | 46.5 | [0.46] | 17110 | 46.3 | [0.33] |
| Age categories (%¹ [SE]) | | | | | | | | | | | | |
| 20-30 y | 1374 | 22.2 | [1.1] | 1089 | 21.6 | [1.2] | 1300 | 22.4 | [0.80] | 3763 | 22.1 | [0.60] |
| 31-50 y | 1619 | 39.9 | [1.9] | 1938 | 39.4 | [1.2] | 2084 | 37.2 | [1.0] | 5641 | 38.8 | [0.82] |
| 51-70 y | 1278 | 26.9 | [1.3] | 1824 | 28.1 | [1.1] | 1854 | 29.4 | [0.94] | 4956 | 28.2 | [0.65] |
| 71+ y | 762 | 10.8 | [1.1] | 1007 | 10.9 | [0.42] | 981 | 11.0 | [0.67] | 2750 | 10.9 | [0.46] |
| Gender (% [SE]) | | | | | | | | | | | | |
| Male | 2416 | 48.1 | [0.49] | 2883 | 48.3 | [0.56] | 3023 | 48.3 | [0.50] | 8322 | 48.3 | [0.30] |
| Female | 2617 | 51.9 | [0.49] | 2975 | 51.9 | [0.56] | 3196 | 51.7 | [0.50] | 8788 | 51.7 | [0.30] |
| Ethnicity (%¹ [SE]) | | | | | | | | | | | | |
| Mexican American | 1048 | 8.04 | [1.0] | 1018 | 8.34 | [1.5] | 1146 | 8.70 | [2.2] | 3212 | 8.40 | [0.95] |
| Non-Hispanic White | 2441 | 71.6 | [2.8] | 2706 | 69.1 | [3.7] | 2962 | 67.7 | [3.3] | 8109 | 69.4 | [1.9] |
| Non-Hispanic Black | 1184 | 11.6 | [1.9] | 1225 | 11.4 | [1.9] | 1138 | 11.5 | [0.86] | 3547 | 11.5 | [0.54] |
| Education (%¹ [SE]) | | | | | | | | | | | | |
| Less than 9th grade | 597 | 6.56 | [0.75] | 763 | 6.86 | [0.73] | 749 | 6.35 | [0.69] | 2109 | 6.59 | [0.42] |
| 9-11th grade | 733 | 11.2 | [1.11] | 1004 | 13.7 | [1.2] | 981 | 12.7 | [0.54] | 2718 | 12.5 | [0.57] |
| High school graduate | 1136 | 25.0 | [0.80] | 1417 | 25.5 | [1.3] | 1389 | 22.8 | [1.13] | 3942 | 24.4 | [0.62] |
| Some college or | 1361 | 31.3 | [0.97] | 1458 | 28.8 | [0.89] | 1696 | 30.4 | [0.79] | 4515 | 30.2 | [0.51] |
| associate's degree | | | | | | | | | | | | |
| College graduate or | 939 | 25.9 | [2.03] | 1059 | 25.2 | [2.0] | 1230 | 27.7 | [1.3] | 3228 | 26.3 | [1.0] |
| above | | | | | | | | | | | | |
| Pregnancy status² (%¹ [SE]) | | | | | | | | | | | | |
| Pregnant | 338 | 5.54 | [0.47] | 57 | 4.07 | [0.68] | 68 | 4.96 | [0.63] | 463 | 4.97 | [0.33] |
| Not pregnant | 1492 | 94.5 | [0.47] | 1096 | 95.9 | [0.68] | 1266 | 95.0 | [0.63] | 3854 | 94.4 | [0.33] |
| Dietary supplement use | | | | | | | | | | | | |
| Yes (%¹ [SE], past 30 d) | 2458 | 53.7 | [1.3] | 2666 | 48.9 | [1.9] | 2885 | 49.5 | 1.2 | 8009 | 50.7 | [0.89] |
| No | 2570 | 46.3 | [1.3] | 3189 | 51.1 | [1.9] | 3330 | 50.5 | 1.2 | 9089 | 49.3 | [0.89] |
| Riboflavin intake (mean | | | | | | | | | | | | |
| [SE], mg/d) | | | | | | | | | | | | |
| Food | 4771 | 2.27 | [0.026] | 5563 | 2.20 | [0.038] | 5918 | 2.16 | [0.028] | 16252 | 2.21 | [0.018] |
| Supplements | 1760 | 8.90 | [0.89] | 1702 | 7.58 | [0.68] | 1770 | 7.45 | [1.0] | 5232 | 8.03 | [0.51] |
| Total | 4835 | 5.88 | [0.38] | 5622 | 4.79 | [0.32] | 5989 | 4.60 | [0.34] | 16446 | 5.08 | [0.20] |
| Fiber intake (mean, | 4771 | 15.9 | [0.22] | 5563 | 16.1 | [0.37] | 5918 | 17.1 | [0.22] | 16252 | 16.4 | [0.16] |
| g/day) | | | | | | | | | | | | |
| Moisture intake from all | 4771 | 3013 | [53] | 5563 | 2884 | [37] | 5918 | 2903 | [31] | 16252 | 2933 | [24] |
| food and beverages | | | | | | | | | | | | |
| (mean [SE], g/day) | | | | | | | | | | | | |
| Laxative use past 30 | | | | | | | | | | | | |
| days (%¹ [SE]) | | | | | | | | | | | | |
| Yes | — | — | | — | — | | 592 | 9.88 | [0.91] | 592 | 9.88 | [0.91] |
| No | — | — | | — | — | | 4679 | 90.1 | [0.91] | 4679 | 90.1 | [0.91] |
| Common stool type (%¹ [SE]) | | | | | | | | | | | | |
| I | 82 | 1.77 | [0.17] | 132 | 2.36 | [0.22] | 106 | 1.75 | [0.14] | 320 | 1.96 | [0.10] |
| II | 237 | 4.38 | [0.33] | 288 | 5.89 | [0.44] | 286 | 4.95 | [0.47] | 811 | 5.08 | [0.25] |
| III | 1140 | 28.1 | [1.27] | 1226 | 25.8 | [0.67] | 1254 | 26.4 | [0.55] | 3620 | 26.7 | [0.51] |
| IV | 2238 | 53.7 | [1.49] | 2628 | 50.8 | [1.5] | 2694 | 51.7 | [1.0] | 7560 | 52.1 | [0.78] |
| V | 281 | 6.37 | [0.42] | 422 | 7.84 | [0.61] | 464 | 8.20 | [0.58] | 1167 | 7.48 | [0.32] |
| VI | 230 | 5.23 | [0.37] | 369 | 6.25 | [0.49] | 389 | 6.40 | [0.36] | 988 | 5.96 | [0.23] |
| VII | 32 | 0.473 | [0.10] | 68 | 1.03 | [0.20] | 53 | 0.619 | [0.093] | 153 | 0.709 | [0.08] |
| Bowel motion frequency | 4294 | 9.1 | [0.08] | 5163 | 9.3 | [0.1] | 5262 | 9.2 | [0.1] | 14719 | 9.2 | [0.06] |
| (mean [SE], bowel | | | | | | | | | | | | |
| motions/week) | | | | | | | | | | | | |
| Normalcy of bowel | | | | | | | | | | | | |
| motion frequency | | | | | | | | | | | | |
| (3-21/w) | | | | | | | | | | | | |
| Normal | 4073 | 95.4 | [0.47] | 4925 | 95.5 | [0.47] | 4990 | 95.3 | [0.38] | 13988 | 95.4 | [0.25] |
| Abnormal | 221 | 4.60 | [0.47] | 238 | 4.51 | [0.47] | 272 | 4.74 | [0.38] | 731 | 4.61 | [0.25] |
| Number of prescription | 5033 | 1.86 | [0.07] | 5858 | 1.92 | [0.08] | 6219 | 1.90 | [0.07] | 17110 | 1.90 | [0.04] |
| medicines (N [SE]) | | | | | | | | | | | | |
| Fecal Incontinence | | | | | | | | | | | | |
| Intensity Score (%¹ [SE]) | | | | | | | | | | | | |
| 0 (least severity) | 2242 | 49.6 | [1.6] | 2853 | 54.2 | [1.14] | 2861 | 53.8 | [1.24] | 7956 | 52.6 | [0.79] |
| 1 to 6 | 629 | 15.4 | [1.3] | 696 | 14.4 | [0.58] | 732 | 15.5 | [0.81] | 2057 | 15.1 | [0.54] |
| 7 to 11 | 685 | 17.2 | [0.89] | 780 | 15.9 | [0.67] | 859 | 16.7 | [0.91] | 2324 | 16.6 | [0.48] |
| 12 and more (greatest | 737 | 17.7 | [0.81] | 814 | 15.5 | [0.81] | 799 | 14.0 | [0.86] | 2350 | 15.7 | [0.49] |
| severity) | | | | | | | | | | | | |

¹Weighted to be representative of the US population.
²Measured in females aged 20 to 44 years.

TABLE S1
Riboflavin dose in dietary supplements
used by U.S. adults aged ≥19 years

| Dietary supplement ribo- | | |
|---|---|---|
| flavin dose (mg/day) | Number of users | % of total |
| 0 to 0.2 | 371 | 5.1 |
| 0.2 to 0.4 | 363 | 5.0 |
| 0.4 to 0.6 | 227 | 3.1 |
| 0.6 to 0.8 | 329 | 4.5 |
| 0.8 to 1 | 500 | 6.9 |
| 1 to 1.2 | 410 | 5.6 |
| 1.2 to 1.4 | 240 | 3.3 |
| 1.4 to 1.6 | 283 | 3.9 |
| 1.6 to 1.8 | 2723 | 37.3 |
| 1.8 to 2 | 30 | 0.4 |
| 2 to 2.2 | 125 | 1.7 |
| 2.2 to 2.4 | 47 | 0.6 |
| 2.4 to 2.6 | 80 | 1.1 |
| 2.6 to 2.8 | 42 | 0.6 |
| 2.8 to 3 | 23 | 0.3 |
| 3 to 4 | 283 | 3.9 |
| 4 to 5 | 85 | 1.2 |
| 5 to 6 | 142 | 1.9 |
| 6 to 7 | 78 | 1.1 |
| 7 to 8 | 13 | 0.2 |
| 8 to 9 | 37 | 0.5 |
| 9 to 10 | 22 | 0.3 |
| 10 to 20 | 234 | 3.2 |
| 20 to 50 | 344 | 4.7 |
| 50 to 100 | 195 | 2.7 |
| 100 to 674 (maximum) | 67 | 0.9 |
| Total | 7293 | 100 |

In Table 2, differences between dietary and supplemental riboflavin are reported. In general, riboflavin intake from dietary supplements is greater than that from the diet and shows greater variability (see supplemental Table S1, above).

TABLE 2
Riboflavin intake from foods, dietary supplements and total according to socio-
demographic and lifestyle characteristics for adults ≥20 y, NHANES 2005-2010

| | Riboflavin intake | | Riboflavin intake | | Total riboflavin | |
|---|---|---|---|---|---|---|
| | from foods | | from dietary | | intake (foods and | |
| Characteristic | (mg/d)* | | supplements (mg/d)* | | supplements, mg/d)* | |
| Entire dataset | 2.16 | [0.016] | 7.06 | [0.44] | 4.38 | [0.15] |
| Gender | | | | | | |
| Male | 2.54 | [0.025]^{a} | 7.68 | [0.62]^{a} | 4.99 | [0.22]^{a} |
| Female | 1.90 | [0.016]^{b} | 8.30 | [0.69]^{a} | 5.18 | [0.29]^{a} |
| Age category | | | | | | |
| 20-30 years | 2.17 | [0.032]^{a} | 6.96 | [0.75]^{a} | 3.96 | [0.23]^{a} |
| 31-50 years | 2.29 | [0.023]^{b} | 7.67 | [0.84]^{a} | 4.87 | [0.29]^{ab} |
| 51-70 years | 2.21 | [0.026]^{ab} | 9.27 | [0.93]^{a} | 6.21 | [0.42]^{b} |
| 70 years and over | 2.00 | [0.024]^{c} | 7.13 | [0.82]^{a} | 5.20 | [0.39]^{ab} |
| Ethnicity | | | | | | |
| Mexican American | 2.02 | [0.026]^{a} | 7.03 | [1.0]^{a} | 5.63 | [0.29]^{a} |
| Non-Hispanic White | 2.34 | [0.017]^{b} | 8.16 | [0.60]^{a} | 3.41 | [0.23]^{b} |
| Non-Hispanic Black | 1.81 | [0.023]^{c} | 7.76 | [0.86]^{a} | 3.74 | [0.25]^{b} |
| Poverty income ratio (PIR) | | | | | | |
| Low (<1.85) | 2.06 | [0.025]^{a} | 6.34 | [0.56]^{a} | 3.63 | [0.15]^{a} |
| Medium (1.85-3.5) | 2.21 | [0.031]^{b} | 7.22 | [0.74]^{a} | 4.78 | [0.25]^{b} |
| High (>3.5) | 2.33 | [0.018]^{c} | 9.27 | [0.88]^{b} | 6.45 | [0.40]^{c} |
| Education | | | | | | |
| Less than 9th grade | 1.84 | [0.048]^{a} | 6.16 | [1.1]^{abc} | 3.06 | [0.26]^{a} |
| 9-11th grade | 2.11 | [0.038]^{b} | 4.61 | [0.53]^{a} | 3.08 | [0.14]^{a} |
| High school graduate | 2.17 | [0.028]^{bc} | 6.95 | [0.78]^{ab} | 4.38 | [0.23]^{b} |
| Some college or AA degree | 2.25 | [0.029]^{bc} | 9.26 | [0.92]^{bc} | 5.87 | [0.40]^{c} |
| College graduate or above | 2.34 | [0.026]^{c} | 8.57 | [0.80]^{bc} | 6.41 | [0.40]^{c} |
| Fiber intake quartiles | | | | | | |
| First (0-8.8 g/d) | 1.51 | [0.028]^{a} | 7.57 | [2.0]^{a} | 3.28 | [0.50]^{a} |
| Second (8.8-12.8 g/d) | 1.86 | [0.016]^{b} | 7.18 | [0.79]^{a} | 4.22 | [0.26]^{q} |
| Third (12.8-18.1 g/d) | 2.20 | [0.025]^{c} | 7.97 | [0.81]^{a} | 5.20 | [0.31]^{ab} |
| Fourth (18.1 g/d or more) | 2.77 | [0.027]^{d} | 8.95 | [0.80]^{a} | 6.42 | [0.35]^{b} |
| Moisture intake quartiles | | | | | | |
| First (0-1382 g/d) | 1.27 | [0.020]^{a} | 6.09 | [1.2]^{a} | 2.65 | [0.30]^{a} |
| Second (1382-1980 g/d) | 1.69 | [0.018]^{b} | 7.48 | [1.7]^{a} | 3.93 | [0.50]^{a} |
| Third (1980-2837 g/d) | 2.07 | [0.021]^{c} | 7.04 | [0.77]^{a} | 4.57 | [0.27]^{ab} |
| Fourth (2837 g/d or more) | 2.63 | [0.021]^{d} | 9.21 | [0.57]^{a} | 6.21 | [0.24]^{b} |
| Dietary supplement use | | | | | | |
| Yes | 2.27 | [0.021]^{a} | 8.03 | [0.51] | 7.81 | [0.36]^{a} |
| No | 2.15 | [0.022]^{b} | — | | 2.15 | [0.022]^{b} |
| Prescription medication count | | | | | | |
| Less than 2 | 2.25 | [0.021]^{a} | 7.98 | [0.52]^{a} | 4.76 | [0.19]^{a} |
| 2 or more | 2.15 | [0.021]^{b} | 8.09 | [0.80]^{a} | 5.57 | [0.35]^{a} |
| Fecal Incontinence Intensity | | | | | | |
| Score | | | | | | |
| 0 (least severity) | 2.21 | [0.021]^{a} | 7.60 | [0.55]^{a} | 4.78 | [0.19]^{a} |
| 1 to 6 | 2.22 | [0.033]^{a} | 7.65 | [1.2]^{a} | 5.31 | [0.49]^{a} |
| 7 to 11 | 2.22 | [0.032]^{a} | 9.20 | [1.3]^{a} | 5.84 | [0.55]^{a} |
| 12 or more (greatest | 2.28 | [0.031]^{a} | 9.28 | [1.5]^{a} | 5.77 | [0.60]^{a} |
| severity) | | | | | | |

*Values with different superscript letters are significantly different

Although women had a lower intake of dietary riboflavin than men, their greater use of dietary supplements containing riboflavin meant that total riboflavin intake was similar. Elderly people aged over 70 had the lowest riboflavin intake from foods.

Non-Hispanic Whites had the highest mean riboflavin intakes from foods, with non-Hispanic Blacks having the lowest, and Mexican Americans intermediate. Mexican Americans had the highest total riboflavin intake.

As Poverty-Income ratio (PIR) increased, indicating an increase in household income, food, supplemental and total riboflavin intakes increased. The same relationship was seen with educational status: as education level increased, so did riboflavin intake via food, supplements and total.

There was also a positive correlation between riboflavin and dietary fiber intakes, with a Pearson Correlation Coefficient of 0.461 (p<0.0001), 0.0867 (p<0.0001) and 0.125 (p<0.0001) for food dietary fiber intake, and food, supplemental and total riboflavin intake, respectively.

Total moisture intake (which includes all moisture from food and beverages, including tap and bottled waters) also increased with increasing riboflavin intakes.

Food riboflavin intake was significantly higher in dietary supplement users, as was total riboflavin intake.

People with lower use of prescription medication (less than the median) had a higher riboflavin intake from food, but supplemental and total riboflavin intake was not different.

While there appeared to be an increasing trend for riboflavin intake and FISI score, this was not significant.

The results of the linear regression are shown in Table 3.

TABLE 3
Beta coefficients from chunk-wise modeling approach for bowel
motion frequency by riboflavin intake and sociodemograhic
and lifestyle factors for adults ≥19 y, NHANES 2005-2010

| Model | Food riboflavin intake | | DS riboflavin intake | | Total riboflavin intake | |
|---|---|---|---|---|---|---|
| Bowel motion | Beta- | | Beta- | | Beta- | |
| frequency | coefficient | p-value | coefficient | p-value | coefficient | p-value |
| Crude model | 0.355 | <0.0001 | 0.0101 | 0.020 | 0.0113 | 0.010 |
| Adjusted model | 0.253 | <0.0001 | 0.0108 | 0.013 | 0.0130 | 0.0041 |
| (demographics^{a}) | | | | | | |
| Adjusted model | 0.0465 | 0.47 | 0.00807 | 0.049 | 0.00684 | 0.075 |
| (diet/health^{b}) | | | | | | |
| Adjusted model | 0.0191 | 0.76 | 0.00895 | 0.030 | 0.00974 | 0.015 |
| (full^{c}) | | | | | | |

^{a}Adjusted for gender, age, education, Poverty-Income Ratio and ethnicity.
^{b}Adjusted for use of prescription medications, self-report of thyroid problem, fiber and moisture intake.
^{c}Adjusted for all variables mentioned above.

There was a significant, positive relationship found between riboflavin intakes from food, dietary supplements and in total, and bowel motion frequency in the crude model. However, after adjusting for socio-demographic, and diet and lifestyle co-factors, the relationship was not significant for food riboflavin although the fully adjusted model showed a significant, positive relationship between riboflavin intake from dietary supplements and in total, and bowel motion frequency.

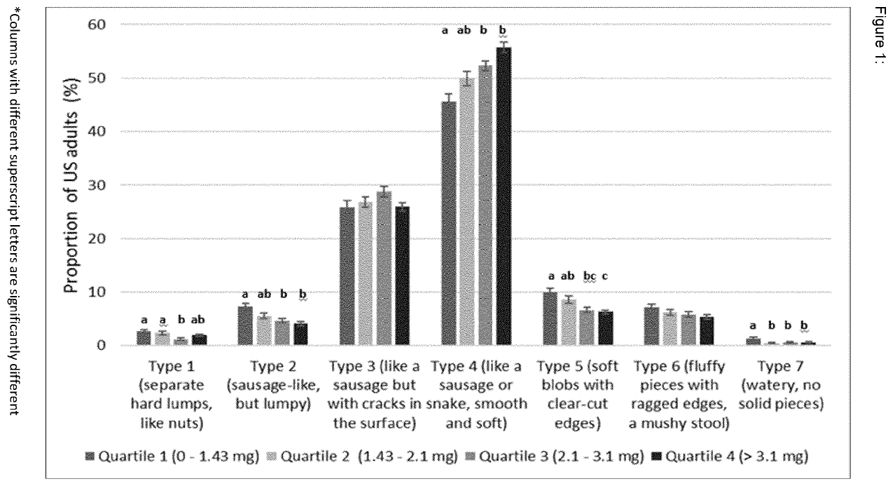

The Bristol Stool Type according to quartiles of riboflavin intake is shown in FIG. 1. As riboflavin intake increases, there is a reduction in stool types 2 (slow transit time) and 5 (soft-fluid stool), and an increase in type 4 (normal defecation).

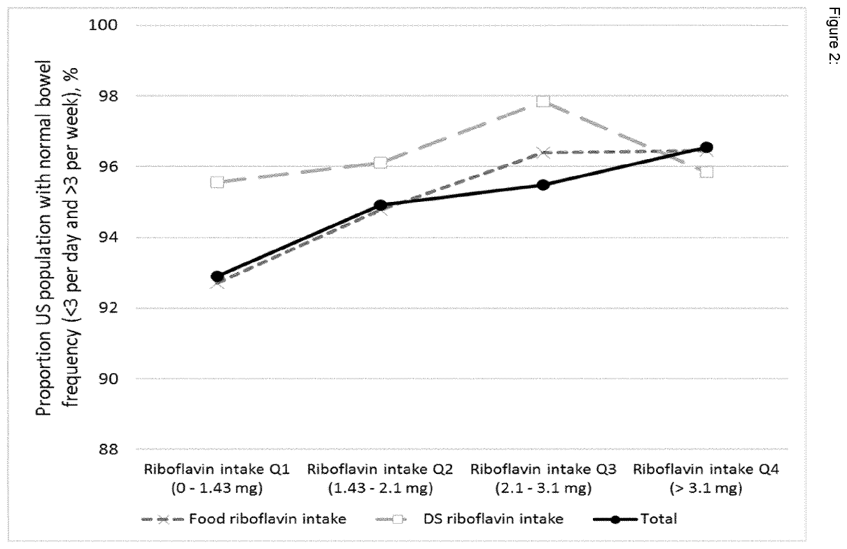

The normalcy of bowel motion frequency as defined as 3-21 bowel motions per week according to riboflavin intake quartiles is depicted in FIG. 2.

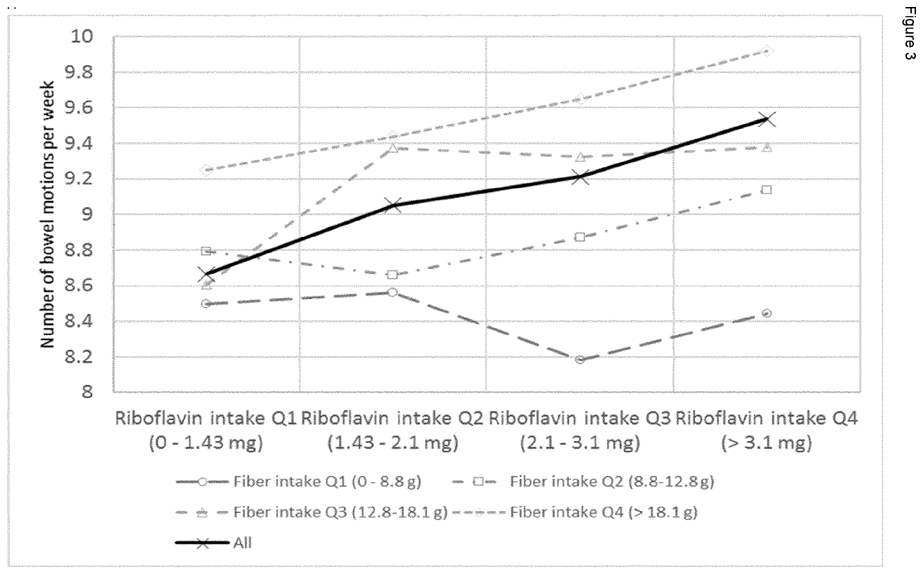

The proportion of the population with normal bowel motion patterns increases as riboflavin intake increases for food and total riboflavin. As there was a particularly high correlation between food and total riboflavin intake, FIG. 3 illustrates the relationship between total riboflavin intake and bowel motion frequency, stratified by fiber intake quartiles. There is an increase in bowel motion frequency as riboflavin intake increases, and stratification for fiber intake shows the same relationship when fiber intakes are higher than the median. Interestingly, the relationship is only significant in the top two quartiles of dietary fiber intake; when dietary fiber intake is low, less than the median, there is no significant relationship between riboflavin intake and bowel motion frequency.

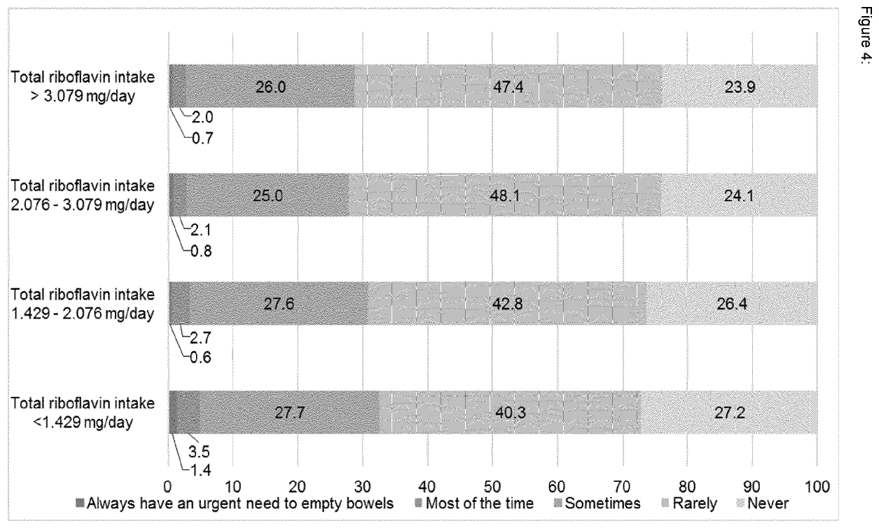

In FIG. 4, the association between bowel motion urgency and riboflavin intake is shown. There is a significant increase in the number of respondents reporting that this occurs “rarely” in the highest two quartiles of riboflavin intake, and this appears to be due to a non-significant decrease in respondents reporting that bowel motion urgency occurs both “never” and more frequently than “rarely”.

### DISCUSSION

Our results show that riboflavin intake from both food and dietary supplements is correlated with improvements in subjective bowel health parameters. The increase in bowel movement frequency with higher riboflavin intakes corresponded to an increase in the proportion of participants with normal stool types (type 4) and a move away from extremes of subjective bowel motion urgency. The increase in bowel movement frequency did not mean a reduction in bowel motion frequency normalcy: a greater proportion of subjects reported being within the “3 and 3” metric of normal frequency at higher intake quartiles.

There is a clear correlation between fiber and riboflavin intake seen in the dataset. While the best dietary sources of riboflavin (organ meats, egg and dairy products) lack dietary fiber, high fiber cereal products, especially whole grains and fortified breakfast cereals, make an important contribution to overall riboflavin intakes. In addition, higher socio-economic status is associated with higher intakes of fiber, and also better dietary quality, resulting in correlated intakes.

Crude and adjusted linear regression showed a significant association between bowel movement frequency and riboflavin intake, from dietary supplements and in total (Table 3). The linear regression adjusted model included dietary fiber as a regressor in the diet/heath chunk, to account for possible confounding. The relationship between bowel movement frequency and riboflavin intake, stratified for fiber intake, was investigated in FIG. 3. From FIG. 3, we saw that bowel movement frequency only increased with riboflavin intake in the top two quartiles of fiber intake.

Our analysis showed that riboflavin intakes generally exceeded the DRIs, and there is little evidence that riboflavin intakes are inadequate in the US. Fulgoni et all 2011 J. Nutrition 141 1847-51 found that less than 5% of the population had inadequate riboflavin intake.

The effect of riboflavin on bowel health parameters was seen when riboflavin intakes were above the median of 2.1 mg/day, and especially in the highest quartile of riboflavin intake (>3.1 mg/d). In subjects consuming a diet containing adequate riboflavin, 1-3% of urinary metabolites recovered are a product of riboflavin degradation by the microbiome which indicates that unabsorbed dietary riboflavin reaches the large intestine, where it is metabolized by intestinal microbes. See, Chastain, 1987 Am J Clin Nutrition 46 830-834. The amount of riboflavin absorbed is dependent on the dose given, with absorption increasing linearly up to a saturable dose of approximately 30 mg (Powers 2003 Am J Clinical Nutrition 77: 1352-1360. Absorption is lower when riboflavin is taken in a fasting state compared with being taken with food, likely due to faster transit time through the proximal small intestine, where most absorption occurs, when the stomach is empty. Higher supplemental doses would therefore result in a larger amount remaining unabsorbed in the gut lumen, and thus available to the gut microbiota. Without wishing to be bound by theory, it appears that a greater amount of riboflavin reaching the colon from supplements may explain why adjusting for diet/health confounders lead to a loss in significance for dietary riboflavin but not for supplemental or total riboflavin.

To our knowledge, this is the first study to report an association between riboflavin intake and bowel health parameters in an observational study in humans. Strengths of our study include the large sample size, and extensive parameters tested as part of the linear regression models. The use of two 24-hour dietary recalls is the gold standard for dietary assessment in a large sample.

Taking the results together, as riboflavin intake increased, the most common stool type changed towards a more normal type and moved away from either diarrhea or constipation (FIG. 1), bowel motion frequency was more likely to be normal (FIG. 2), and bowel motion urgency normalized (FIG. 4) as riboflavin intake increased.

1. Riboflavin for the non-therapeutic use of promoting a bowel health parameter in a healthy person selected from the group consisting of:
A) normalizing diarrhea or constipation;
B) regulating bowel movement frequency; and
C) regulating bowel movement urgency.

2. Riboflavin according to claim 1 wherein the total amount of riboflavin consumed by diet and by supplement is above the recommended daily amount (RDA) for the healthy person.

3. Riboflavin according to claim 1 where the amount of riboflavin consumed in a supplement is at least 1 to 5 mg, preferably 1 to 3 mgs, and more preferably, at least 1.5 to 2 mg.

4. Riboflavin according to claim 1, in combination with dietary fibre.

5. A method of promoting bowel health in a healthy person comprising: administering an effective amount of riboflavin to a person experiencing, or at risk of experiencing, at least one condition selected from the group consisting of: diarrhea, constipation, irregular bowel movement frequency, and irregular bowel movement urgency.

6. The method according to claim 5, wherein a total amount of riboflavin consumed by diet and by supplement is above the recommended daily amount for the healthy person.

7. The method according to claim 6 wherein the amount of riboflavin consumed by supplement is at least 1 to 5 mg, preferably 1. to 3 mgs, and more preferably, at least 1.5 to 2 mg.

8. The method according to claim 5, further comprising administering dietary fibre.
